(19) United States
(12) Patent Application Publication
(10) Pub. No.: US 2021/0386274 A1
(43) Pub. Date: Dec. 16, 2021

(54) ENT GUIDE WITH ADVANCEABLE INSTRUMENT AND ADVANCEABLE ENDOSCOPE SHAFT

(71) Applicants: Acclarent, Inc., Irvine, CA (US); Biosense Webster (Israel) Ltd., Yokneam (IL)

(72) Inventors: Jetmir Palushi, Irvine, CA (US); Yehuda Algawi, Binyamina (IL); Assaf Govari, Haifa (IL); Fatemeh Akbarian, Rancho Palos Verdes, CA (US); Itzhak Fang, Irvine, CA (US); Athanasios Papadakis, Newport Beach, CA (US); Madison K. Vanosdoll, Irvine, CA (US)

(21) Appl. No.: 17/324,201

(22) Filed: May 19, 2021

Related U.S. Application Data
(60) Provisional application No. 63/037,640, filed on Jun. 11, 2020.

(51) Int. Cl. A61B 1/005; A61B 17/00; A61B 17/24; A61B 1/05; A61B 10/04; A61B 18/14; A61M 29/02; A61B 1/018; A61B 1/00

(52) U.S. Cl. CPC A61B 1/0057; A61B 17/00234; A61B 17/24; A61B 1/05; A61B 10/04; A61B 2018/00577; A61M 29/02; A61B 1/018; A61B 1/00147; A61B 1/0052; A61B 18/14

(57) ABSTRACT

An apparatus includes a body, a shaft assembly, and a shaft actuation assembly. The shaft assembly extends distally from the body and defines a longitudinal axis. A portion of the shaft assembly is configured to be inserted into an anatomical passageway associated with an ear, nose, or throat of a patient. The shaft assembly further defines a working passageway that is dimensioned to receive an instrument. An actuator of the body is operable to drive the instrument longitudinally relative to the shaft assembly. The shaft actuation assembly is operatively coupled with a proximal portion of the shaft assembly. Further, the shaft actuation assembly is translatable in a distal direction to extend the shaft assembly distally along the longitudinal axis and is translatable in a proximal direction to retract the shaft assembly proximally along the longitudinal axis.

### PRIORITY

This application claims priority to U.S. Provisional Pat. App. No. 63/037,640, entitled “ENT Guide with Advanceable Instrument and Advanceable Endoscope Shaft,” filed Jun. 11, 2020, the disclosure of which is incorporated by reference herein.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, now abandoned, the disclosure of which is incorporated by reference herein. An example of such a system is the Relieva® Spin Balloon Sinuplasty System by Acclarent, Inc. of Irvine, Calif.

In the context of Eustachian tube dilation, a dilation catheter or other dilation instrument may be inserted into the Eustachian tube and then be inflated or otherwise expanded to thereby dilate the Eustachian tube. The dilated Eustachian tube may provide improved ventilation from the nasopharynx to the middle ear and further provide improved drainage from the middle ear to the nasopharynx. Methods and devices for dilating the Eustachian tube are disclosed in U.S. Patent Pub. No. 2010/0274188, entitled “Method and System for Treating Target Tissue within the ET,” published on Oct. 28, 2010, now abandoned, the disclosure of which is incorporated by reference herein; and U.S. Patent Pub. No. 2013/0274715, entitled “Method and System for Eustachian Tube Dilation,” published on Oct. 17, 2013, now abandoned, the disclosure of which is incorporated by reference herein. An example of such a system is the Aera® Eustachian Tube Balloon Dilation System by Acclarent, Inc. of Irvine, Calif.

It may be desirable to provide easily controlled placement of a dilation catheter or other ENT instrument in an anatomical passageway, including in procedures that will be performed only by a single operator. While several systems and methods have been made and used to position a dilation catheter or other ENT instrument in an anatomical passageway, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1A depicts a perspective view of an exemplary guide instrument for use in ear, nose, and throat (ENT) procedures, with a guide slider and an instrument slider each in respective proximal positions;

FIG. 1B depicts a perspective view of the instrument of FIG. 1A, with the guide slider in a distal position and the instrument slider in the proximal position, and with the guide shaft in a longitudinally extended state;

FIG. 1C depicts a perspective view of the instrument of FIG. 1A, with the guide slider and the instrument slider each in respective distal positions, with the guide shaft in a longitudinally extended state, and with an instrument longitudinally extended from the guide tip;

FIG. 2 depicts a perspective view of a guide shaft assembly of the instrument of FIG. 1A, with the flex section shown in solid lines in a non-deflected configuration, and with the flex section shown in phantom lines deflecting away from the longitudinal axis of the guide shaft assembly;

FIG. 3A depicts a side view of the flex section of the instrument of FIG. 1A, with the flex section in a non-deflected state;

FIG. 3B depicts a side view of the flex section of the instrument of FIG. 1A, with the flex section in a deflected state;

FIG. 4 depicts a perspective view of the guide shaft assembly of the instrument of FIG. 1A, with the flex section shown in solid lines in a first angular position, and with the flex section shown in phantom lines rotated about the longitudinal axis of the guide shaft assembly to a second angular position;

FIG. 5 depicts a perspective view of the distal end of the guide shaft assembly of the instrument of FIG. 1A, with a camera positioned at the distal tip of the flex section;

FIG. 6 depicts an exploded perspective view of the distal end of the guide shaft assembly of the instrument of FIG. 1A;

FIG. 7A depicts a perspective view of the distal end of the guide shaft assembly of the instrument of FIG. 1A, with a first exemplary instrument extending outward from the distal tip of the flex section;

FIG. 7B depicts a perspective view of the distal end of the guide shaft assembly of the instrument of FIG. 1A, with a second exemplary instrument extending outward from the distal tip of the flex section; and

FIG. 7C depicts a perspective view of the distal end of the guide shaft assembly of the instrument of FIG. 1A, with a third exemplary instrument extending outward from the distal tip of the flex section.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. For example, while various. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

### I. EXEMPLARY ENT GUIDE INSTRUMENT

A. Overview

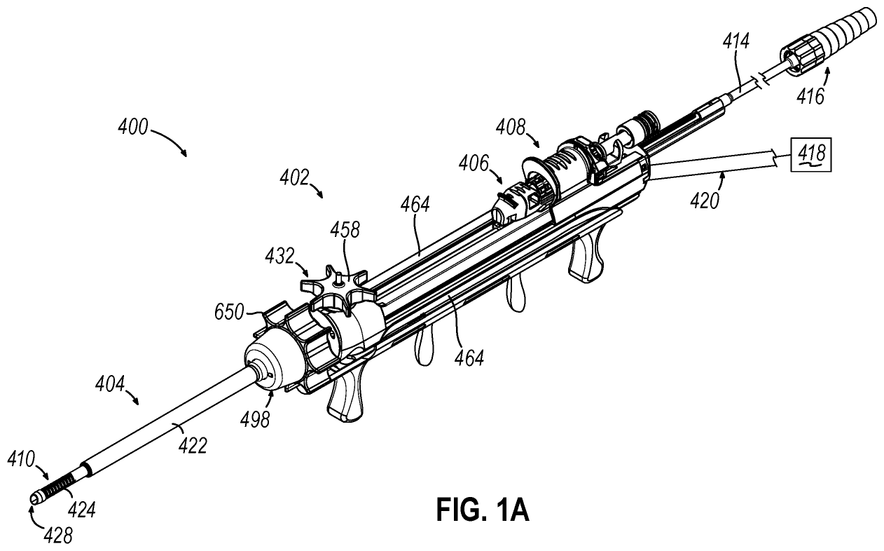

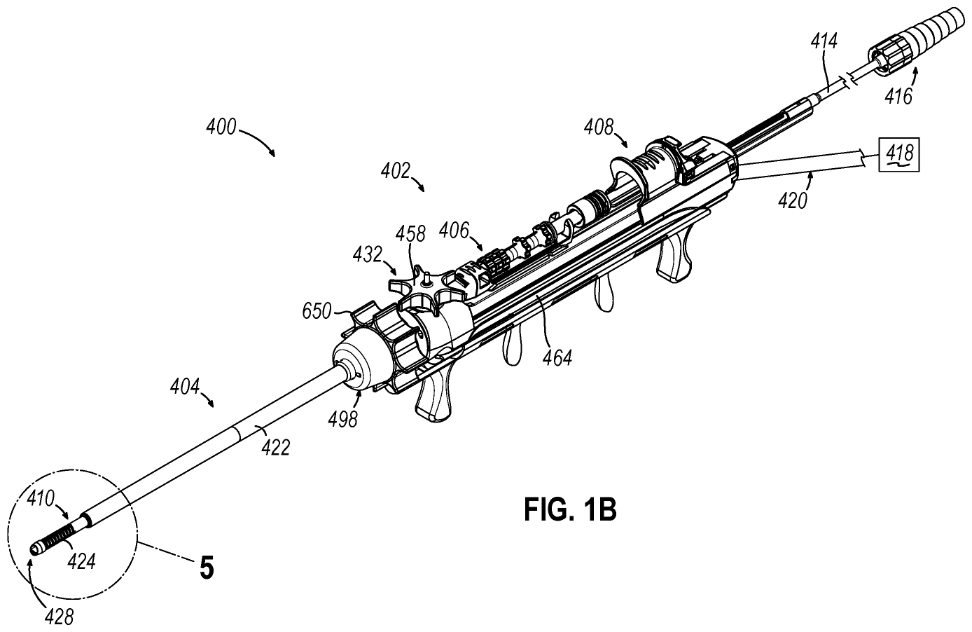

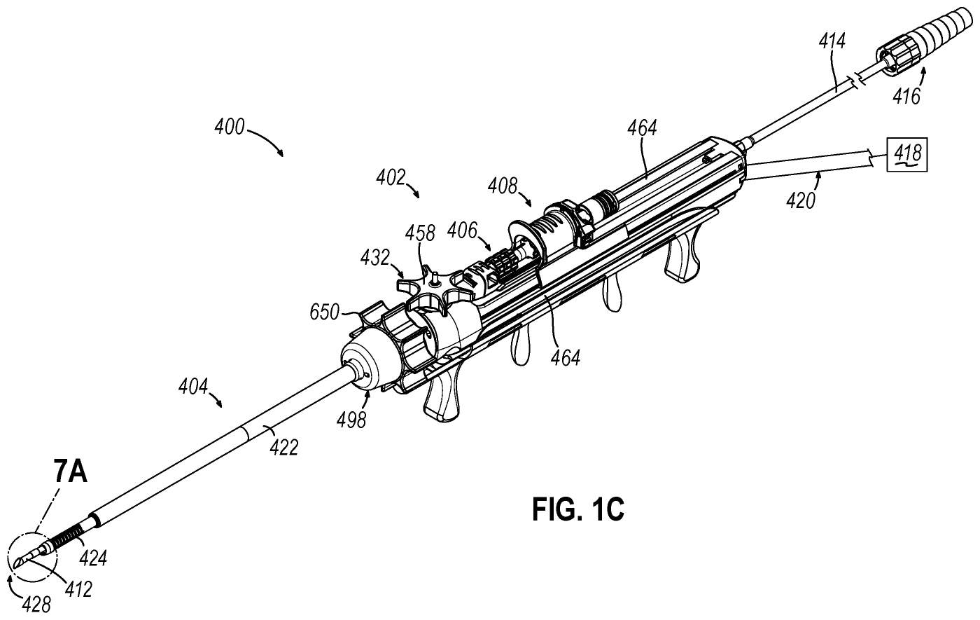

FIGS. 1A-1C show an exemplary ENT guide instrument (400) that may be used for medical procedures within the ear, nose, or throat. Such procedures may include ablation within the nasal cavity; dilation of an ostium of a paranasal sinus , a Eustachian tube, or some other anatomical passageway (e.g., within the ear, nose, or throat, etc.); suctioning of fluids or debris from within the nasal cavity or ear; performing a biopsy within an ear, nose, or throat; removing bone or tissue from within the nasal cavity; or any other suitable procedure within the ear, nose, or throat as will be apparent to those skilled in the art in view of the teachings herein. As will be described in greater detail below, ENT guide instrument (400) of the present example provides adjustability that enables the operator to use ENT guide instrument (400) in different scenarios, without requiring the operator to switch between different instruments. For instance, ENT guide instrument (400) may be used to access various different anatomical passageways by making simple adjustments to structural features of the instrument; and then address those different anatomical passageways with various ENT instrumentation that is coupled with ENT guide instrument (400).

B. Exemplary Guide Shaft Actuation Assembly

ENT guide instrument (400) of this example includes a handle assembly (402) having a housing (464), a guide shaft assembly (404) extending distally from handle assembly (402); a guide shaft actuation assembly, or guide slider (406), which is slidably coupled with handle assembly (402); and an instrument slider (408) slidably coupled with handle assembly (402). One or more instruments (412) (see FIG. 1C) may be inserted into guide shaft assembly (404) of ENT guide instrument (400) via port (416) and through inner shaft (414), which spans through handle assembly (402). In the present example, inner shaft (414) is coaxially disposed within guide shaft assembly (404), and instrument (412) is coaxially disposed within inner shaft (414). A suction source (418) is coupled with guide shaft assembly (404) of ENT guide instrument (400) via a suction port (420) and a conduit (not shown) which spans through handle assembly (402). In some versions, suction port (420) is omitted.

Handle assembly (402) is sized and configured to be grasped and operated by a single hand of an operator. The operator may selectively operate guide slider (406) or instrument slider (408), with the same single hand that grasps handle assembly (402). The operator may translate guide slider (406) distally along handle assembly (402) to thereby telescopically translate guide shaft assembly (404) such that the distal end (410) of guide shaft assembly (404) longitudinally translates distally away from handle assembly (402). The operator may also translate guide slider (406) proximally along handle assembly (402) to thereby telescopically translate guide shaft assembly (404) such that the distal end (410) of guide shaft assembly (404) longitudinally retracts proximally toward handle assembly (402). Thus, guide shaft assembly (404) may be effectively longitudinally lengthened or shortened during operation, as needed, prior to or during the operation.

For instance, as shown in FIG. 1A, guide slider (406) is translated to its proximal-most position relative to handle assembly (402), and similarly, guide shaft assembly (404) is retracted to its shortest effective length configuration. As shown in the transition from FIG. 1A to FIG. 1B, guide slider (406) is translated distally relative to handle assembly (402), thereby telescopically extending the effective length of guide shaft assembly (404).

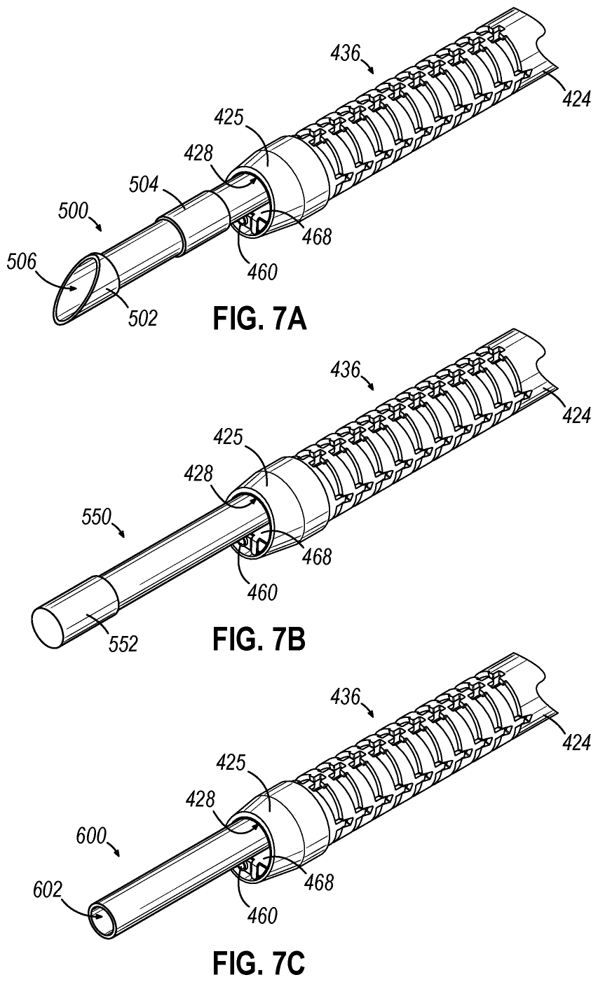

As shown in the transition from FIG. 1B to FIG. 1C, the operator may advance instrument slider (408) distally along handle assembly (402) to thereby advance an instrument (412) distally through inner shaft (414) and guide shaft assembly (404), such that the distal tip of instrument (412) is translated through an opening (428) and distal to the distal end of guide shaft assembly (404). With instrument (412) advanced to a distal position, the operator may then utilize instrument (412) within an anatomical passageway in which instrument (412) is positioned. Various examples of instruments are described herein with respect to FIG. 7A to FIG. 7C; while other examples will be apparent to those skilled in the art in view of the teachings herein. In some versions, a guidewire or dilation catheter, such as a catheter having an expandable dilator, are included in place of instrument (412). Example guidewires and expandable dilators are disclosed in U.S. Patent Pub. No. 2010/0274188, entitled “Method and System for Treating Target Tissue within the ET,” published on Oct. 28, 2010, now abandoned, the disclosure of which is incorporated by reference herein.

While FIGS. 1B and 1C show instrument slider (408) being actuated distally when guide slider (406) is advanced to the distal-most position to advance guide shaft assembly (404) to the maximum effective length, in other scenarios the operator may wish to actuate instrument slider (408) distally when guide slider (406) is in the proximal-most position shown in FIG. 1A with guide shaft assembly (404) being at a minimum effective length (or when guide slider (406) is in some other, intermediate longitudinal position). Thus, the actuation of instrument slider (408) is not necessarily dependent on the longitudinal positioning of guide slider (406) or guide shaft assembly (404).

By way of example only, an operator may wish to operate guide instrument (400) with guide slider (406) in the distal-most position, with guide shaft assembly (404) at the maximum effective length, when guide instrument (400) will be inserted in a nasal cavity of a patient. By way of further example only, an operator may wish to operate guide instrument (400) with guide slider (406) in the proximal-most position, with guide shaft assembly (404) at the minimum effective length, when guide instrument (400) will be inserted in an ear canal of a patient. Other usage scenarios that may influence the selection of effective length for guide shaft assembly (404) will be apparent to those skilled in the art in view of the teachings herein. Similarly, the selections of types of instruments that may be coupled with guide instrument (400) and actuated by instrument slider (408) based on the usage scenario at hand will be apparent to those skilled in the art in view of the teachings herein.

C. Exemplary Deflection and Shaft Rotation Actuation Assemblies

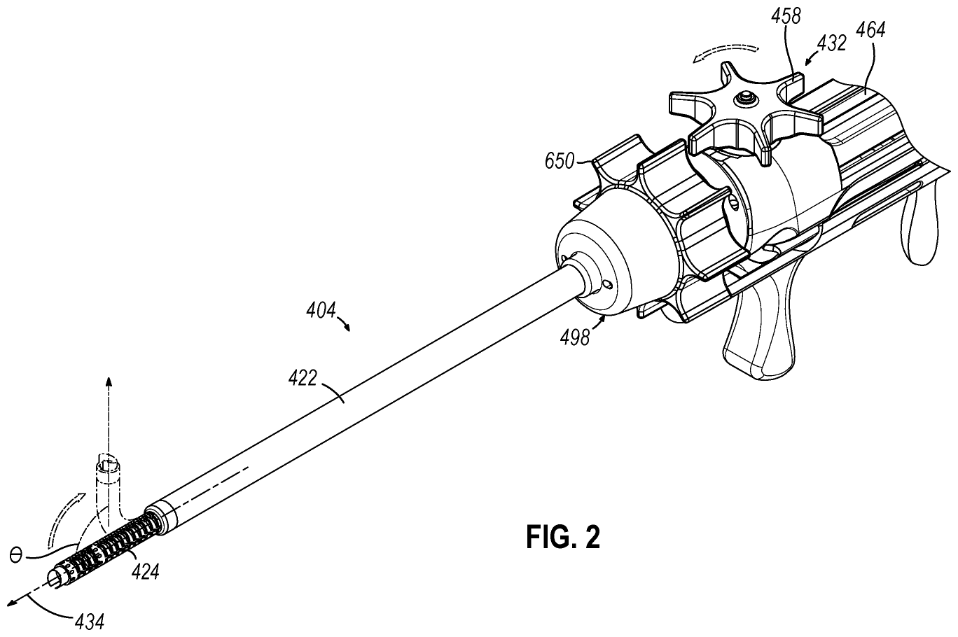

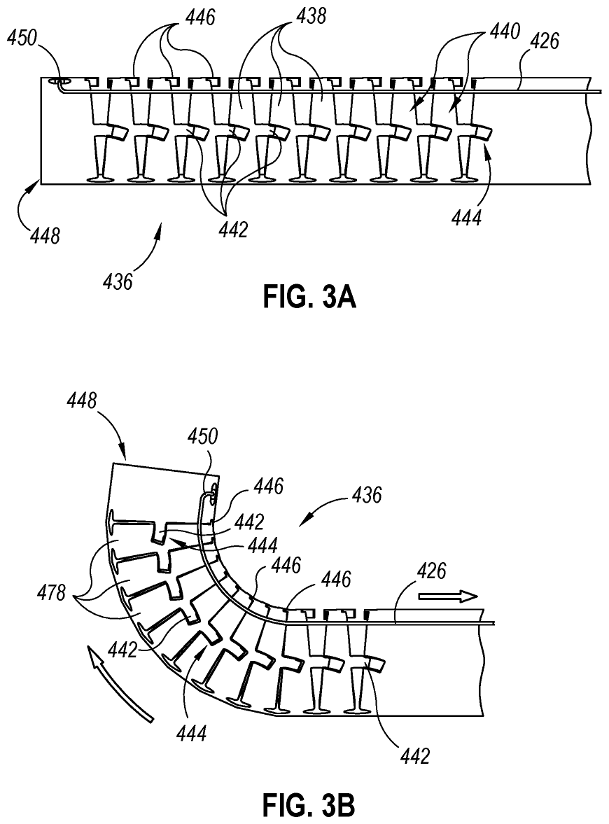

FIGS. 2-4 show various components of guide shaft assembly (404) in greater detail. As described above, guide shaft assembly (404) includes a rigid shaft member (422) and a flexible shaft member (424). Additionally, guide shaft assembly (404) includes a push-pull wire (426) (see FIGS. 3A and 3B) and rotary actuators, such as deflection control assembly (432) and shaft rotation assembly (498). Shaft rotation assembly (498) includes a thumbwheel (650). Shaft members (422, 424) and thumbwheel (650) are coaxially aligned with each other in this example, and thumbwheel (650) is configured to rotate about longitudinal axis (434) to thereby rotate guide shaft assembly (404) about longitudinal axis (434).

Deflection control assembly (432) includes a thumbwheel (458) configured to rotate about an axis transverse to longitudinal axis (434), for example, perpendicular to longitudinal axis (434). Push-pull wire (426) is operatively coupled with thumbwheel (458) and is laterally offset from the longitudinal axis (434) shared by shaft members (422, 424) and shaft rotation assembly (498). As described in greater detail below, push-pull wire (426) is operable to longitudinally translate within guide shaft assembly (404) and thereby deflect flexible shaft member (424) away from or toward longitudinal axis (434); and thumbwheel (458) is operable to drive translation of push-pull wire (426).

Thumbwheels (458, 650) are positioned such that an operator may rotate thumbwheels (458, 650) relative to housing (464) using the thumb or another finger of the hand that is grasping handle assembly (402). In some versions, the positions of deflection control assembly (432) and shaft rotation assembly (498) (including thumbwheels (458, 650)) may be switched such that thumbwheel (458) of deflection control assembly (432) is configured to rotate about longitudinal axis (434) and thumbwheel (650) of shaft rotation assembly (498) is configured to rotate about an axis transverse to longitudinal axis (434). As will be described in greater detail below, guide shaft assembly (404) is operable to guide one or more instruments along an operator-selected exit angle relative to the central longitudinal axis (434) of guide shaft assembly (404).

In some versions, both shaft members (422, 424) are formed of a metallic material, such as stainless steel and/or nitinol. In some such versions, shaft members (422, 424) (and at least some other portions of ENT guide instrument (400)) may be reusable, with such reusable components being subject to cleaning and sterilization between uses on different patients. In some other versions, one or both of shaft members (422, 424) may be formed of a polymeric material. In some such versions, shaft members (422, 424) may be treated as single-use-only components. Flexible shaft member (424) is fixedly secured to rigid shaft member (422) in this example and is positioned distally in relation to rigid shaft member (422).

As best seen in FIGS. 3A-3B, flexible shaft member (424) includes a flex section (436) that is formed by a series of ribs (438), which are separated by a series of notches (440). Notches (440) are generally V-shaped, with a circular opening at the vertex of each “V.” Notches (440) also include tab portions (442) that fit in corresponding sub-notches (444). The top of each “V” includes a set of stop features (446). As shown in FIG. 3A, when flex section (436) is in a straight configuration, tab portions (442) are disposed in corresponding sub-notches (444) but are not fully seated in sub-notches (444). As also shown in FIG. 3A, when flex section (436) is in a straight configuration, stop features (446) are separated from each other. FIG. 3B shows flex section (436) in a fully bent configuration. In this state, tab portions (442) are fully seated in sub-notches (444) and stop features (446) are engaged with each other. During the transition between the states shown in FIGS. 3A-3B, tab portions (442) and sub-notches (444) may cooperate to ensure that flex section (436) bends in a consistent fashion, with sufficient lateral stability; and that flex section (436) provides a consistent and stable bent or straight state.

By way of example only, flex section (436) may be formed through laser cutting or any other suitable manufacturing process. In some versions, flex section (436) is covered with a flexible wrap (not shown). Such a flexible wrap may prevent tissue and other structures from getting snagged or pinched in notches (440), without compromising the flexibility of flex section (436). A flexible wrap may also ensure that suction provided through guide shaft assembly (404) is focused at distal end (448). Various suitable forms that flex section (436) may take will be apparent to those of ordinary skill in the art in view of the teachings herein. By way of further example only, flex section (436) may be constructed and operable in accordance with at least some of the teachings of U.S. Pat. Pub. No. 2018/0311472, entitled “Deflectable Guide for Medical Instrument,” published Nov. 1, 2018, the disclosure of which is incorporated by reference herein.

Push-pull wire (426) is disposed within shaft members (422, 424) and is operable to provide controlled bending of flex section (436). As shown in FIGS. 3A-3B, a distal end (450) of push-pull wire (426) is secured to the distal end (448) of flexible shaft member (424), distal to flex section (436). Push-pull wire (426) is disposed near the tops of the “V”s of notches (440). Thus, when push-pull wire (426) is pulled proximally, flex section (436) will bend to a deflected configuration. When push-pull wire (426) is pushed distally, flex section (436) will bend toward a straight configuration. A proximal end of push-pull wire (426) operatively couples with thumbwheel (458) of deflection control assembly (432). As is illustrated in FIG. 2, rotation of thumbwheel (458) causes longitudinal translation of push-pull wire (426), and longitudinal translation of push-pull wire (426) thereby causes straightening or bending of flex section (436) about angle (θ). Various suitable ways in which thumbwheel (458) may be coupled with push-pull wire (426) to provide the operability described above will be apparent to those skilled in the art in view of the teachings herein.

While flex section (436) is actively steered in the present example via thumbwheel (458) and push-pull wire (426) other versions may include a flex section (436) that is simply malleable. In such versions, the operator may simply bend flex section (436) manually (e.g., using a separate bending template or other tool) before inserting guide shaft assembly (404) into the patient. In such versions, thumbwheel (458) and push-pull wire (426) may be omitted from guide instrument (400).

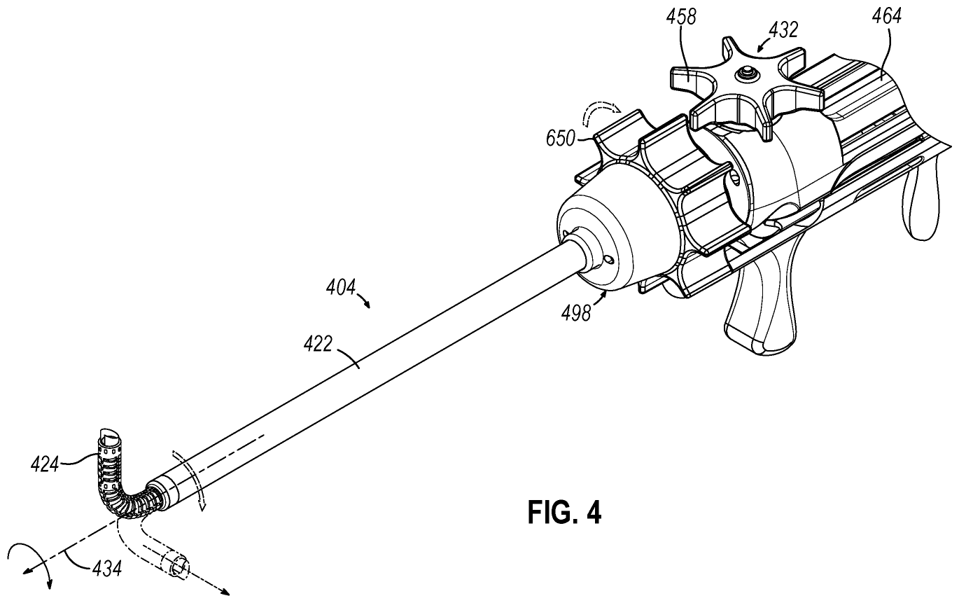

In addition to providing control for the deflection of flex section (436) to facilitate access to various anatomical passageways, it may be desirable to enable rotation of guide shaft assembly (404) about the longitudinal axis (434) of guide shaft assembly (434), to further facilitate access to various anatomical passageways. To that end, as shown in FIG. 4, ENT guide instrument (400) includes shaft rotation assembly (498) for rotating guide shaft assembly (404) about the longitudinal axis (434). Guide shaft assembly (404) rotation can be performed concurrently with deflection control assembly (432), guide slider (406), and instrument slider (408), if so desired. For example, an operator may require performance of series of guide shaft assembly (404) adjustments, including both rigid shaft member (422) rotation and flexible shaft member (424) deflection to reach the desired anatomical passageway; as well as adjusting the length of rigid shaft member (422) via guide slider (406) and the positioning of instrument (412) at the distal end of flexible shaft member (424) via instrument slider (408).

D. Exemplary ENT Instruments for Use with Guide Instrument

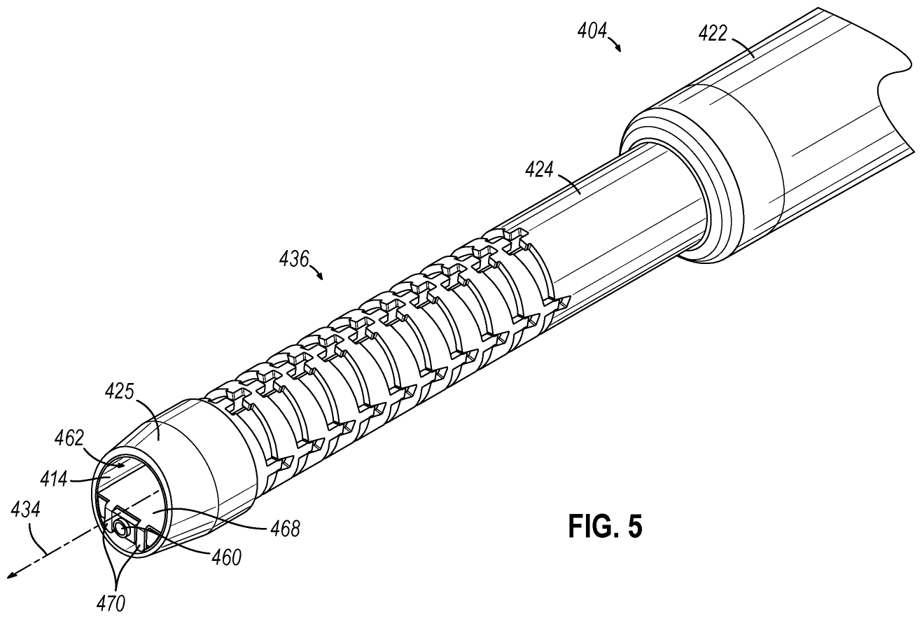

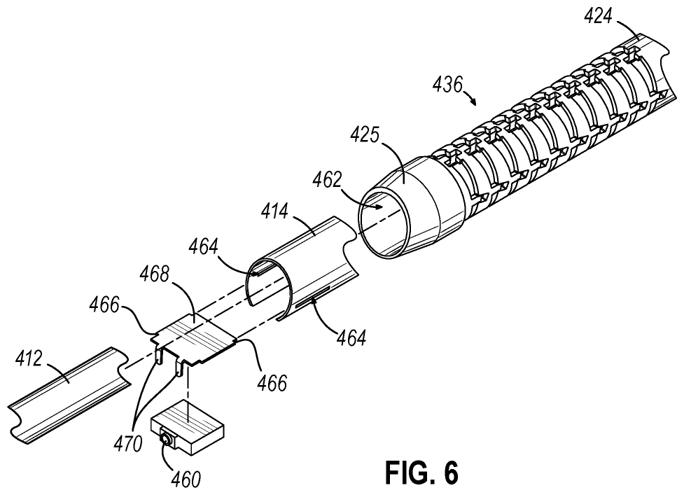

FIGS. 5-6 show various components and functional aspects of flexible shaft member (424) of guide shaft assembly (404) in greater detail. As described above, flexible shaft member (424) includes a push-pull wire (426) (see FIGS. 3A and 3B) that is operable to deflect flex section (436) of flexible shaft member (424) away from longitudinal axis (434). In some versions, a camera (460) is positioned integrally at the distal tip (425) of the flexible shaft member (424) and is configured to provide an operator with a field of view (FOV) adjacent the distal tip (425). An instrument may be advanced through the working instrument channel (462) and out of the opening (428) at the distal tip (425) of flexible shaft member (424). Distal tip (425) may be comprised of a plastic or other similar material and includes a tapered configuration to facilitate insertion of guide shaft assembly (404) through tight anatomical passageways. Camera (460) may include a live-feed video camera operatively coupled with a video monitor mounted in view of the operator to convey real time video images from the camera (460) for display on the monitor. Camera (460) may include power and communications wiring (not shown) routed through inner shaft (414) of guide shaft assembly (404) to directly couple with a power source and video monitor. In some variations, camera (460) may be battery operated and configured for wireless communication with the video monitor.

As best shown in FIG. 6, the distal end of flexible shaft member (424) includes various features to ensure camera (460) remains properly positioned during an operation, and also remains spatially separated from and prevented from blocking working instrument channel (462). For example, as noted above, inner shaft (414) extends through flexible shaft member (424) and terminates adjacent the distal tip of flexible shaft member (424). In some versions, inner shaft (414) includes slots (464) shaped to accept tabs (466) formed along opposing edges of a separator (468) to prevent longitudinal or transverse displacement. Thus, as inner shaft (414) longitudinally translates in conjunction with rigid shaft member (422), separator (468) remains positioned at the distal tip of flexible shaft member (424). In some versions, camera (460) is fixedly secured to the bottom surface of separator (468) at the distal tip of flexible shaft member (424), while in other versions camera (460) is translatable within inner shaft (414) through handle assembly (402) and shaft assembly (404) by an operator and is steerable toward placement against the bottom surface of separator (468). To ensure camera (460) is restricted from exiting instrument working channel (462) at the distal tip of flexible shaft member (424), separator (468) includes legs (470) that are transversely oriented to abut distal engagement surfaces of camera (460). With separator (468) in place, the distal tip of instrument working channel (462) includes an open passageway for an instrument (412) to pass through while also providing a secure position for camera (460) to view and record the movements of instrument (412).

FIGS. 7A-7C depict various exemplary instruments (500, 550, 600) that may be utilized in place of instrument (412) as described herein. FIG. 7A depicts a biopsy needle (500) that is configured to translate through shaft assembly (404) and extend through opening (428) of the instrument working channel (462). Biopsy needle (500) of this example includes a pair of electrodes (502, 504) that are operable to apply bipolar radiofrequency (RF) coagulation of the puncture tract as biopsy needle (500) punctures tissue and collects a tissue sample within hollowed needle cavity (506). FIG. 7B depicts an ablation instrument (550) that is configured to translate through shaft assembly (404) and extend through opening (428) of the instrument working channel (462). Ablation instrument (550) may include an electrode (552) that is operable to apply monopolar RF energy to ablate tissue. Alternatively, ablation instrument (550) may include two or more electrodes (552) to apply bipolar RF energy to ablate tissue. FIG. 7C depicts a suction instrument (600) that is configured to translate through shaft assembly (404) and extend through opening (428) of the instrument working channel (462). Suction instrument (600) of this example includes a lumen (602) open at the distal end that is operable to suction matter therethrough. Lumen (602) is fluidly coupled with suction source (418) and suction port (420). In some variations, suction may be applied directly via working instrument channel (462), such that a separate suction instrument (600) is not necessarily required in order to apply suction via ENT guide instrument (400).

While various exemplary instrument versions have been described herein, it should be understood that additional instruments may also be utilized with ENT guide instrument (400). For example, guidewires and catheters including expandable dilation balloons may also be utilized as required by the particular circumstances of the operation being performed. Other suitable kinds of ENT instruments that may be used with ENT guide instrument (400) will be apparent to those skilled in the art in view of the teachings herein.

Regardless of the kind of ENT instrument that is coupled with instrument slider (408) and fed through guide shaft assembly (404), the adjustability of guide shaft assembly (404) (e.g., adjustment of effective length via guide slider (406), adjustment of bend angle of flex section (436) via thumbwheel (432), and adjust/Andent of rotational angle of guide shaft assembly (404) via thumbwheel (650)) may facilitate guidance of the ENT instrument to the appropriate location within the patient. Moreover, the video image provided by camera (460) may provide real time visualization that further facilitates guidance of the ENT instrument to the appropriate location within the patient. With camera (460) being integrated into guide shaft assembly (404), ENT guide instrument (400) may occupy less space within anatomical passageways of the patient than might otherwise be occupied by the combination of a conventional guide tube and conventional endoscope (e.g., in a side-by-side positioning). In addition, the integration of camera (460) into ENT guide instrument (400) may further promote single-handed operation of both ENT guide instrument (400) and whichever ENT instrument is coupled with instrument slider (408) and fed through guide shaft assembly (404), since the operator will not need to also grasp a separate endoscope.

### II. EXEMPLARY COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a body; (b) a shaft assembly extending distally from the body and defining a longitudinal axis, wherein a portion of the shaft assembly is configured to be inserted into an anatomical passageway associated with an ear, nose, or throat of a patient, wherein the shaft assembly defines a working passageway that is dimensioned to slidably receive an instrument; (c) an instrument actuator coupled with the body, wherein the instrument actuator is operable to drive an instrument longitudinally relative to the shaft assembly while the instrument is disposed in the working passageway; and (d) a shaft actuation assembly operatively coupled with a proximal portion of the shaft assembly, wherein the shaft actuation assembly is translatable in a distal direction to extend the shaft assembly distally along the longitudinal axis relative to the body, wherein shaft actuation assembly is translatable in a proximal direction to retract the shaft assembly proximally along the longitudinal axis relative to the body.

### Example 2

The apparatus of Example 1, wherein the shaft actuation assembly is slidably coupled with the body, wherein the shaft actuation assembly is configured to translate longitudinally relative to the body.

### Example 3

The apparatus of any of Examples 1-2, wherein a distal portion of the shaft assembly includes a flexible section.

### Example 4

The apparatus of Example 3, further comprising a deflection actuation assembly, wherein the deflection actuation assembly includes a translatable actuation member extending through the shaft assembly, wherein the translatable actuation member is operatively coupled with the flexible section of the shaft assembly and is thereby operable to flex the flexible section of the shaft assembly.

### Example 5

The apparatus of Example 4, further comprising a first rotary actuator, wherein the first rotary actuator is rotatable by a rotational force to thereby drive the translatable actuation member longitudinally, wherein the flexible section is configured to deflect away from the longitudinal axis in response to translation of the translatable actuation member longitudinally.

### Example 6

The apparatus of any of Examples 4-5, wherein the translatable actuation member includes a pull-wire.

### Example 7

The apparatus of any of Examples 4-6, wherein the first rotary actuator is configured to rotate about a rotary axis, wherein the rotary axis is oriented transverse to the longitudinal axis.

### Example 8

The apparatus of any of Examples 1-7, further comprising a camera positioned at a distal end of the shaft assembly.

### Example 9

The apparatus of any of Examples 1-8, further comprising an instrument positioned within the working passageway, wherein the instrument is longitudinally slidable relative to the shaft assembly.

### Example 10

The apparatus of Example 9, further comprising an instrument actuation assembly operatively coupled with the body, wherein the instrument actuation assembly is operable to translate the instrument distally through the working passageway.

### Example 11

The apparatus of any of Examples 9-10, wherein the instrument includes a suction instrument.

### Example 12

The apparatus of any of Examples 9-10, wherein the instrument includes a biopsy needle.

### Example 13

The apparatus of any of Examples 9-10, wherein the instrument includes an ablation instrument.

### Example 14

The apparatus of any of Examples 9-10, wherein the instrument includes a dilation catheter, wherein the dilation catheter comprises an expandable dilator.

### Example 15

The apparatus of any of Examples 1-14, further comprising a shaft rotation assembly, wherein the shaft rotation assembly is operable to rotate the shaft assembly about the longitudinal axis.

### Example 16

The apparatus of Example 15, wherein the shaft rotation assembly comprises a rotary actuator positioned at a distal portion of the body, wherein the rotary actuator is rotatable about the longitudinal axis.

### Example 17

An apparatus, comprising: (a) a body; (b) a shaft assembly extending distally from the body and defining a longitudinal axis, wherein a portion of the shaft assembly is configured to be inserted into an anatomical passageway accessible via an ear, nose, or throat of a patient, wherein the shaft assembly includes a flexible distal portion, wherein the shaft assembly defines a working passageway that is dimensioned to slidably receive an instrument; (c) a shaft actuation assembly, wherein the shaft actuation assembly is operable to translate the shaft assembly longitudinally relative to the body; and (d) a deflection actuation assembly, wherein the deflection actuation assembly includes a translatable actuation member extending through the shaft assembly, wherein the translatable actuation member is operatively coupled with the flexible distal portion of the shaft assembly.

### Example 18

The apparatus of Example 17, wherein the shaft actuation assembly is slidably coupled with the body, wherein the shaft actuation assembly is configured to translate longitudinally relative to the body.

### Example 19

The apparatus of any of Examples 17-18, further comprising a first rotary actuator, wherein the first rotary actuator is rotatable by a rotational force to thereby drive the translatable actuation member longitudinally, wherein the flexible distal portion is configured to deflect away from the longitudinal axis in response to translation of the translatable actuation member longitudinally.

### Example 20

The apparatus of Example 19, wherein the first rotary actuator is configured to rotate about a rotary axis, wherein the rotary axis is oriented transverse to the longitudinal axis.

### Example 21

The apparatus of any of Examples 17-20, further comprising a camera positioned at a distal end of the flexible distal portion.

### Example 22

The apparatus of any of Examples 17-21, further comprising an instrument positioned within the instrument channel, wherein the instrument is longitudinally slidable relative to the shaft assembly.

### Example 23

The apparatus of Example 22, further comprising an instrument actuation assembly operatively coupled with a proximal portion of the shaft assembly, wherein the instrument actuation assembly is operable to translate the instrument distally through the instrument channel.

### Example 24

The apparatus of any of Examples 22-23, wherein the instrument includes a suction instrument.

### Example 25

The apparatus of any of Examples 22-23, wherein the instrument includes a biopsy needle.

### Example 26

The apparatus of any of Examples 22-23, wherein the instrument includes an ablation instrument.

### Example 27

The apparatus of any of Examples 22-23, wherein the instrument includes a dilation catheter, wherein the dilation catheter comprises an expandable dilator.

### Example 28

The apparatus of any of Examples 17-27, further comprising a shaft rotation assembly, wherein the shaft rotation assembly is operable to rotate the shaft assembly about the longitudinal axis.

### Example 29

The apparatus of Example 28, wherein the shaft rotation assembly comprises a rotary actuator positioned at a distal portion of the body, wherein the rotary actuator is rotatable about the longitudinal axis.

### Example 30

The apparatus of any of Examples 17-29, wherein the translatable actuation member includes a pull-wire.

### Example 31

An apparatus, comprising: (a) a body; (b) a shaft assembly extending distally from the body and defining a longitudinal axis, wherein a portion of the shaft assembly is configured to be inserted into an anatomical passageway that is accessible via an ear, nose, or throat of a patient, wherein the shaft assembly defines a working passageway that is dimensioned to slidably receive an instrument; (c) a shaft actuation assembly, wherein the shaft actuation assembly is operable to translate the shaft assembly longitudinally relative to the body; and (d) a shaft rotation assembly, wherein the shaft rotation assembly is operable to rotate the shaft assembly about the longitudinal axis.

### Example 32

The apparatus of Example 31, wherein the shaft actuation assembly is translatable in a distal direction to advance the shaft assembly distally along the longitudinal axis relative to the body, wherein shaft actuation assembly is translatable in a proximal direction to retract the shaft assembly proximally along the longitudinal axis relative to the body.

### Example 33

The apparatus of any of Examples 31-32, wherein the shaft actuation assembly is slidably coupled with the body, wherein the shaft actuation assembly is configured to translate longitudinally relative to the body.

### III. MISCELLANEOUS

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus, comprising:
(a) a body;
(b) a shaft assembly extending distally from the body and defining a longitudinal axis, wherein a portion of the shaft assembly is configured to be inserted into an anatomical passageway associated with an ear, nose, or throat of a patient, wherein the shaft assembly defines a working passageway that is dimensioned to slidably receive an instrument;
(c) an instrument actuator coupled with the body, wherein the instrument actuator is operable to drive an instrument longitudinally relative to the shaft assembly while the instrument is disposed in the working passageway; and
(d) a shaft actuation assembly operatively coupled with a proximal portion of the shaft assembly, wherein the shaft actuation assembly is translatable in a distal direction to extend the shaft assembly distally along the longitudinal axis relative to the body, wherein shaft actuation assembly is translatable in a proximal direction to retract the shaft assembly proximally along the longitudinal axis relative to the body.

2. The apparatus of claim 1, wherein the shaft actuation assembly is slidably coupled with the body, wherein the shaft actuation assembly is configured to translate longitudinally relative to the body.

3. The apparatus of claim 1, wherein a distal portion of the shaft assembly includes a flexible section.

4. The apparatus of claim 3, further comprising a deflection actuation assembly, wherein the deflection actuation assembly includes a translatable actuation member extending through the shaft assembly, wherein the translatable actuation member is operatively coupled with the flexible section of the shaft assembly and is thereby operable to flex the flexible section of the shaft assembly.

5. The apparatus of claim 4, further comprising a first rotary actuator, wherein the first rotary actuator is rotatable by a rotational force to thereby drive the translatable actuation member longitudinally, wherein the flexible section is configured to deflect away from the longitudinal axis in response to translation of the translatable actuation member longitudinally.

6. The apparatus of claim 4, wherein the translatable actuation member includes a pull-wire.

7. The apparatus of claim 4, wherein the first rotary actuator is configured to rotate about a rotary axis, wherein the rotary axis is oriented transverse to the longitudinal axis.

8. The apparatus of claim 1, further comprising a camera positioned at a distal end of the shaft assembly.

9. The apparatus of claim 1, further comprising an instrument positioned within the working passageway, wherein the instrument is longitudinally slidable relative to the shaft assembly.

10. The apparatus of claim 9, further comprising an instrument actuation assembly operatively coupled with the body, wherein the instrument actuation assembly is operable to translate the instrument distally through the working passageway.

11. The apparatus of claim 9, wherein the instrument includes a suction instrument.

12. The apparatus of claim 9, wherein the instrument includes a biopsy needle.

13. The apparatus of claim 9, wherein the instrument includes an ablation instrument.

14. The apparatus of claim 9, wherein the instrument includes a dilation catheter, wherein the dilation catheter comprises an expandable dilator.

15. The apparatus of claim 1, further comprising a shaft rotation assembly, wherein the shaft rotation assembly is operable to rotate the shaft assembly about the longitudinal axis.

16. The apparatus of claim 15, wherein the shaft rotation assembly comprises a rotary actuator positioned at a distal portion of the body, wherein the rotary actuator is rotatable about the longitudinal axis.

17. An apparatus, comprising:
(a) a body;
(b) a shaft assembly extending distally from the body and defining a longitudinal axis, wherein a portion of the shaft assembly is configured to be inserted into an anatomical passageway accessible via an ear, nose, or throat of a patient, wherein the shaft assembly includes a flexible distal portion, wherein the shaft assembly defines a working passageway that is dimensioned to slidably receive an instrument;
(c) a shaft actuation assembly, wherein the shaft actuation assembly is operable to translate the shaft assembly longitudinally relative to the body; and
(d) a deflection actuation assembly, wherein the deflection actuation assembly includes a translatable actuation member extending through the shaft assembly, wherein the translatable actuation member is operatively coupled with the flexible distal portion of the shaft assembly.

18. The apparatus of claim 17, wherein the shaft actuation assembly is slidably coupled with the body, wherein the shaft actuation assembly is configured to translate longitudinally relative to the body.

19. The apparatus of claim 17, further comprising a first rotary actuator, wherein the first rotary actuator is rotatable by a rotational force to thereby drive the translatable actuation member longitudinally, wherein the flexible distal portion is configured to deflect away from the longitudinal axis in response to translation of the translatable actuation member longitudinally.

20. An apparatus, comprising:
(a) a body;
(b) a shaft assembly extending distally from the body and defining a longitudinal axis, wherein a portion of the shaft assembly is configured to be inserted into an anatomical passageway that is accessible via an ear, nose, or throat of a patient, wherein the shaft assembly defines a working passageway that is dimensioned to slidably receive an instrument;
(c) a shaft actuation assembly, wherein the shaft actuation assembly is operable to translate the shaft assembly longitudinally relative to the body; and
(d) a shaft rotation assembly, wherein the shaft rotation assembly is operable to rotate the shaft assembly about the longitudinal axis.
